(19) United States
(12) Patent Application Publication
(10) Pub. No.: US 2017/0342100 A1
(43) Pub. Date: Nov. 30, 2017

(54) PROCESSES FOR THE PREPARATION OF ERTUGLIFLOZIN

(71) Applicant: SUN PHARMACEUTICAL INDUSTRIES LIMITED, Mumbai, Maharashtra (IN)

(72) Inventors: Israr ALI, South Delhi (IN); Rajesh KUMAR, Unnao (IN); Dhiren Chandra BARMAN, Guwahati (IN); Asok NATH, Gurgaon (IN); Mohan PRASAD, Gurgaon (IN)

(21) Appl. No.: 15/532,555

(22) PCT Filed: Dec. 3, 2015
(86) PCT No.: PCT/IB2015/059333

§ 371 Date: Jun. 2, 2017

(30) Foreign Application Priority Data
Dec. 3, 2014 (IN) 3518/DEL/2014

(51) Int. Cl. C07H 9/04; C07H 15/04; C07F 7/18; C07H 15/207; C07D 309/10

(52) U.S. Cl. CPC C07H 9/04; C07H 15/207; C07D 309/10; C07F 7/1856; C07H 15/04

(57) ABSTRACT

The present invention relates to processes for the preparation of ertugliflozni. The present invention also provides compounds of Formula (III), Formula (IV), and Formula (VII), processes for their preparation, and their use for the preparation of ertugliflozin. The processes of the present invention involve protecting the ertugliflozin intermediate compound with a suitable protecting group which provides ertugliflozin having high purity and yield.

### FIELD OF THE INVENTION

The present invention relates to processes for the preparation of ertugliflozin. The present invention also provides compounds of Formula III, Formula IV, and Formula VII, processes for their preparation, and their use for the preparation of ertugliflozin.

### BACKGROUND OF THE INVENTION

Ertugliflozin, chemically (1S,2S,3S,4R,5S)-5-{4-chloro-3-[(4-ethoxyphenyl)methyl]phenyl}-1-(hydroxymethyl)-6,8-dioxabicyclo[3.2.1]octane-2,3,4-triol, is represented by Formula I.

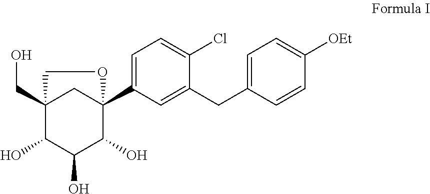 [SMILES: CCOc1ccc(Cc2cc([C@@]34C[C@@](CO)(CO3)[C@@H](O)[C@H](O)[C@H]4O)ccc2Cl)cc1]

Ertugliflozin is a selective sodium glucose cotransporter 2 inhibitor for the treatment of type 2 diabetes mellitus.

U.S. Pat. No. 8,080,580 discloses processes for the preparation of ertugliflozin which involves protecting the primary alcohol moiety of an intermediate compound with a trityl group in the presence of pyridine and subsequent deprotection with p-toluenesulfonic acid. This patent also discloses conversion of the ertugliflozin to ertugliflozin L-pyroglutamic acid.

PCT Publication No. WO 2014/159151 discloses a process for the preparation of ertugliflozin and its conversion to ertugliflozin L-pyroglutamic acid.

The deprotection of the trityl group in the presence of an acid catalyst leads to low purity and yield of the product. Also, pyridine is a hazardous chemical and its use for the manufacture of a drug product is not advisable.

### SUMMARY OF THE INVENTION

The present invention provides processes for the preparation of ertugliflozin. The present invention also provides compounds of Formula III, Formula IV, and Formula VII, processes for their preparation, and their use for the preparation of ertugliflozin. The processes of the present invention involve protecting the ertugliflozin intermediate compound with a suitable protecting group which provides ertugliflozin having high purity and yield.

### DETAILED DESCRIPTION OF THE INVENTION

Various embodiments and variants of the present invention are described hereinafter.

The term “about,” as used herein, refers to any value which lies within the range defined by a number up to ±10% of the value.

The term “TBDMSO,” as used herein, refers to “tert-butyldimethylsilyloxy” group.

The term “solvent,” as used herein, includes, for example, saturated or unsaturated hydrocarbons, alcohols, ethers, halogenated hydrocarbons, carboxylic acids, ketones, amides, sulphoxides, water, or mixtures thereof.

Examples of saturated or unsaturated hydrocarbons include benzene, toluene, cyclohexane, and xylenes. Examples of alcohols include methanol, ethanol, 1-propanol, 1-butanol, 2-butanol, and tertiary alcohols having from one to six carbon atoms. Examples of ethers include diethyl ether, ethyl methyl ether, diisopropyl ether, tetrahydrofuran, 2-methyltetrahydrofuran, and 1,4-dioxane. Examples of halogenated hydrocarbons include dichloromethane and chloroform. Examples of carboxylic acids include formic acid, acetic acid, and propionic acid. Examples of ketones include acetone, diethyl ketone, ethyl methyl ketone, and methyl iso-butyl ketone. Examples of amides include N,N-dimethylformamide and N,N-dimethylacetamide. Examples of sulphoxides include dimethyl sulphoxide and diethyl sulphoxide.

The term “base,” as used herein, includes, for example, inorganic and organic bases. Examples of inorganic bases include hydroxides, carbonates, and bicarbonates of alkali and alkaline earth metals, ammonia, and sodium hydride. Examples of alkali and alkaline earth metal hydroxides include lithium hydroxide, sodium hydroxide, potassium hydroxide, magnesium hydroxide, calcium hydroxide, and barium hydroxide. Examples of alkali and alkaline earth metal carbonates include sodium carbonate, potassium carbonate, calcium carbonate, and magnesium carbonate. Examples of alkali metal bicarbonates include sodium bicarbonate and potassium bicarbonate. Examples of organic bases include N,N-diisopropylethylamine, pyridine, triethylamine, triisopropylamine, methylamine, N,N-2-trimethyl-2-propanamine, N-methylmorpholine, 4-dimethylamino-pyridine, 2,6-di-tert-butyl-4-dimethylamino-pyridine, 1,4-diazabicyclo[2.2.2]octane, and 1,8-diazabicyclo[5.4.0]undec-7-ene

A first aspect of the present invention provides a process for the preparation of ertugliflozin of Formula I,

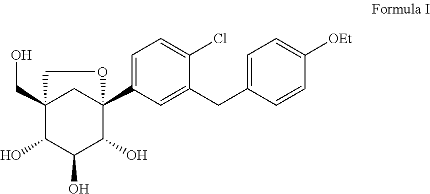 [SMILES: CCOc1ccc(Cc2cc([C@@]34C[C@@](CO)(CO3)[C@@H](O)[C@H](O)[C@H]4O)ccc2Cl)cc1]

wherein the process comprises:
a) protecting a compound of Formula II

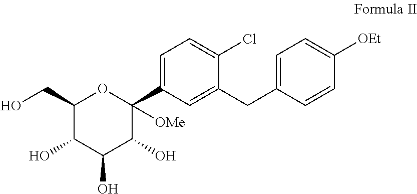 [SMILES: CCOc1ccc(Cc2cc([C@]3(OC)O[C@H](CO)[C@@H](O)[C@H](O)[C@H]3O)ccc2Cl)cc1]

with tert-butyldimethylsilyl chloride to obtain a compound of Formula III;

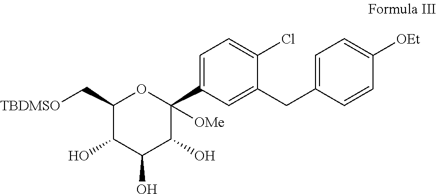 [SMILES: CCOc1ccc(Cc2cc([C@]3(OC)O[C@H](CO[Si](C)(C)C(C)(C)C)[C@@H](O)[C@H](O)[C@H]3O)ccc2Cl)cc1]

b) protecting the compound of Formula III with benzyl bromide to obtain a compound of Formula IV;

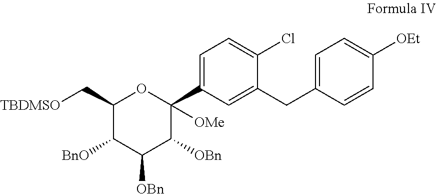 [SMILES: CCOc1ccc(Cc2cc([C@]3(OC)O[C@H](CO[Si](C)(C)C(C)(C)C)[C@@H](OCc4ccccc4)[C@H](OCc4ccccc4)[C@H]3OCc3ccccc3)ccc2Cl)cc1]

c) deprotecting the compound of Formula IV to obtain a compound of Formula V;

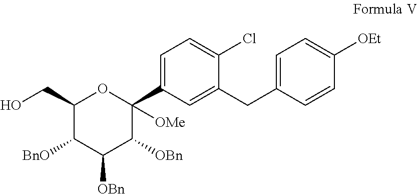 [SMILES: CCOc1ccc(Cc2cc([C@]3(OC)O[C@H](CO)[C@@H](OCc4ccccc4)[C@H](OCc4ccccc4)[C@H]3OCc3ccccc3)ccc2Cl)cc1]

d) oxidizing the compound of Formula V to obtain a compound of Formula VI;

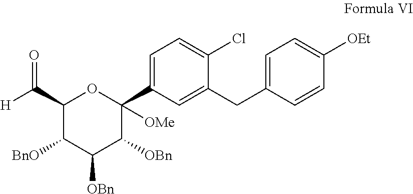 [SMILES: CCOc1ccc(Cc2cc([C@]3(OC)O[C@H](C=O)[C@@H](OCc4ccccc4)[C@H](OCc4ccccc4)[C@H]3OCc3ccccc3)ccc2Cl)cc1]

e) reacting the compound of Formula VI with formaldehyde in the presence of a base to obtain a compound of Formula VII, a compound of Formula VIII, or a mixture thereof;

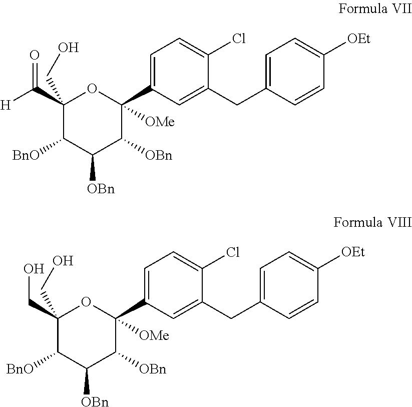

f) optionally, reducing the compound of Formula VII to obtain the compound of Formula VIII;
g) cyclizing the compound of Formula VIII to obtain a compound of Formula IX; and

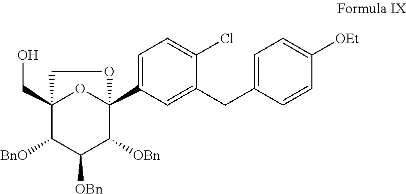 [SMILES: CCOc1ccc(Cc2cc([C@]34OC[C@](CO)(O3)[C@@H](OCc3ccccc3)[C@H](OCc3ccccc3)[C@H]4OCc3ccccc3)ccc2Cl)cc1]

h) debenzylating the compound of Formula IX to obtain ertugliflozin of Formula I.

A second aspect of the present invention provides a process for the preparation of a compound of Formula III,

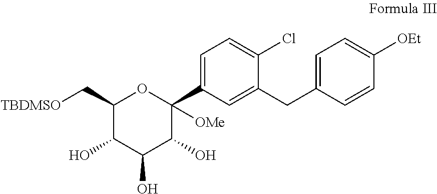 [SMILES: CCOc1ccc(Cc2cc([C@]3(OC)O[C@H](CO[Si](C)(C)C(C)(C)C)[C@@H](O)[C@H](O)[C@H]3O)ccc2Cl)cc1]

wherein the process comprises protecting a compound of Formula II

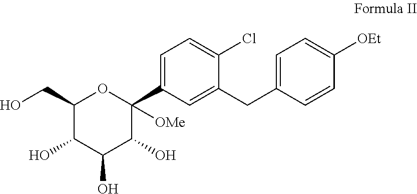 [SMILES: CCOc1ccc(Cc2cc([C@]3(OC)O[C@H](CO)[C@@H](O)[C@H](O)[C@H]3O)ccc2Cl)cc1]

with tert-butyldimethylsilyl chloride to obtain the compound of Formula III.

A third aspect of the present invention provides a process for the preparation of ertugliflozin of Formula I,

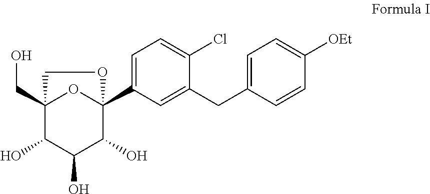 [SMILES: CCOc1ccc(Cc2cc([C@]34OC[C@](CO)(O3)[C@@H](O)[C@H](O)[C@H]4O)ccc2Cl)cc1]

wherein the process comprises:
a) protecting a compound of Formula II

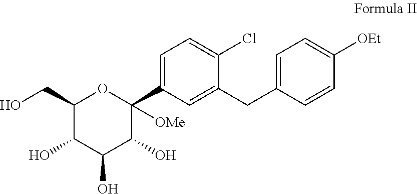 [SMILES: CCOc1ccc(Cc2cc([C@]3(OC)O[C@H](CO)[C@@H](O)[C@H](O)[C@H]3O)ccc2Cl)cc1]

with tert-butyldimethylsilyl chloride to obtain a compound of Formula III; and

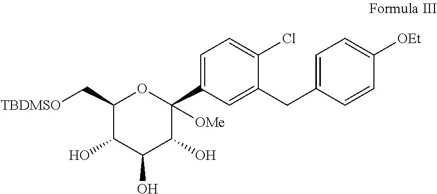 [SMILES: CCOc1ccc(Cc2cc([C@]3(OC)O[C@H](CO[Si](C)(C)C(C)(C)C)[C@@H](O)[C@H](O)[C@H]3O)ccc2Cl)cc1]

b) converting the compound of Formula III to ertugliflozin of Formula I.

A fourth aspect of the present invention provides a process for the preparation of a compound Formula IV,

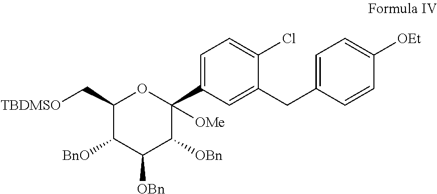 [SMILES: CCOc1ccc(Cc2cc([C@]3(OC)O[C@H](CO[Si](C)(C)C(C)(C)C)[C@@H](OCc4ccccc4)[C@H](OCc4ccccc4)[C@H]3OCc3ccccc3)ccc2Cl)cc1]

wherein the process comprises protecting a compound of Formula III

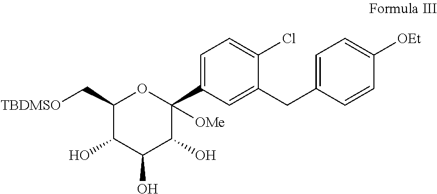 [SMILES: CCOc1ccc(Cc2cc([C@]3(OC)O[C@H](CO[Si](C)(C)C(C)(C)C)[C@@H](O)[C@H](O)[C@H]3O)ccc2Cl)cc1]

with benzyl bromide to obtain the compound of Formula IV.

A fifth aspect of the present invention provides a process for the preparation of ertugliflozin of Formula I,

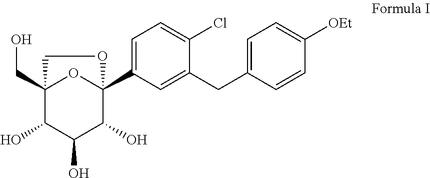 [SMILES: CCOc1ccc(Cc2cc([C@]34OC[C@](CO)(O3)[C@@H](O)[C@H](O)[C@H]4O)ccc2Cl)cc1]

wherein the process comprises:
a) protecting a compound of Formula III

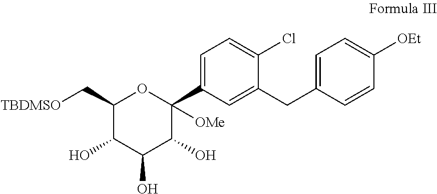 [SMILES: CCOc1ccc(Cc2cc([C@]3(OC)O[C@H](CO[Si](C)(C)C(C)(C)C)[C@@H](O)[C@H](O)[C@H]3O)ccc2Cl)cc1]

with benzyl bromide to obtain a compound of Formula IV; and

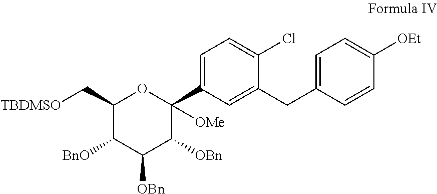 [SMILES: CCOc1ccc(Cc2cc([C@]3(OC)O[C@H](CO[Si](C)(C)C(C)(C)C)[C@@H](OCc4ccccc4)[C@H](OCc4ccccc4)[C@H]3OCc3ccccc3)ccc2Cl)cc1]

b) converting the compound of Formula IV to ertugliflozin of Formula I.

A sixth aspect of the present invention provides a process for the preparation of a compound of Formula IV,

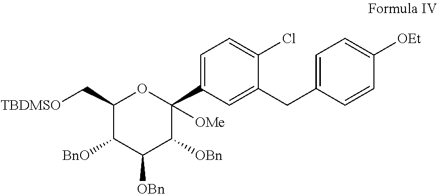 [SMILES: CCOc1ccc(Cc2cc([C@]3(OC)O[C@H](CO[Si](C)(C)C(C)(C)C)[C@@H](OCc4ccccc4)[C@H](OCc4ccccc4)[C@H]3OCc3ccccc3)ccc2Cl)cc1]

wherein the process comprises:
a) protecting a compound of Formula II

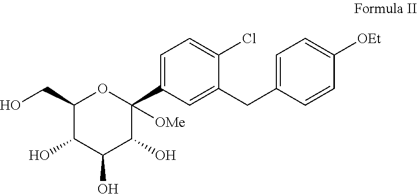 [SMILES: CCOc1ccc(Cc2cc([C@]3(OC)O[C@H](CO)[C@@H](O)[C@H](O)[C@H]3O)ccc2Cl)cc1]

with tert-butyldimethylsilyl chloride to obtain a compound of Formula III; and

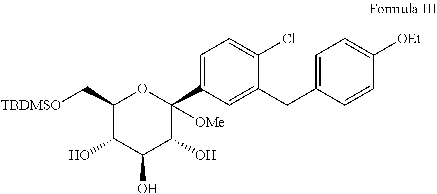 [SMILES: CCOc1ccc(Cc2cc([C@]3(OC)O[C@H](CO[Si](C)(C)C(C)(C)C)[C@@H](O)[C@H](O)[C@H]3O)ccc2Cl)cc1]

b) protecting the compound of Formula III with benzyl bromide to obtain the compound of Formula IV.

A seventh aspect of the present invention provides a process for the preparation ertugliflozin of Formula I,

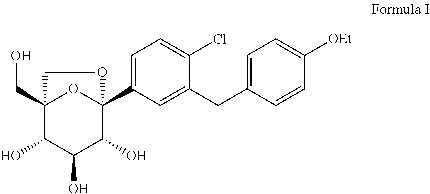 [SMILES: CCOc1ccc(Cc2cc([C@]34OC[C@](CO)(O3)[C@@H](O)[C@H](O)[C@H]4O)ccc2Cl)cc1]

wherein the process comprises:
a) protecting a compound of Formula II

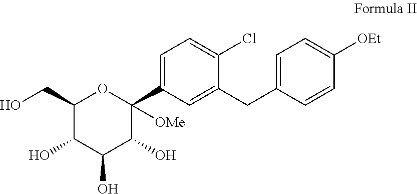 [SMILES: CCOc1ccc(Cc2cc([C@]3(OC)O[C@H](CO)[C@@H](O)[C@H](O)[C@H]3O)ccc2Cl)cc1]

with tert-butyldimethylsilyl chloride to obtain a compound of Formula III;

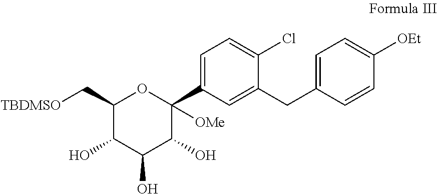 [SMILES: CCOc1ccc(Cc2cc([C@]3(OC)O[C@H](CO[Si](C)(C)C(C)(C)C)[C@@H](O)[C@H](O)[C@H]3O)ccc2Cl)cc1]

b) protecting the compound of Formula III with benzyl bromide to obtain a compound of Formula IV; and

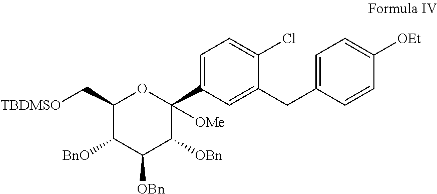 [SMILES: CCOc1ccc(Cc2cc([C@]3(OC)O[C@H](CO[Si](C)(C)C(C)(C)C)[C@@H](OCc4ccccc4)[C@H](OCc4ccccc4)[C@H]3OCc3ccccc3)ccc2Cl)cc1]

c) converting the compound of Formula IV to ertugliflozin of Formula I.

An eighth aspect of the present invention provides a process for the preparation of compound Formula V,

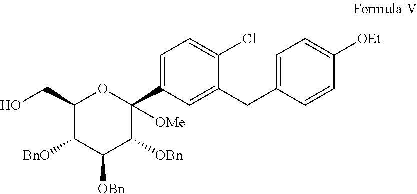 [SMILES: CCOc1ccc(Cc2cc([C@]3(OC)O[C@H](CO)[C@@H](OCc4ccccc4)[C@H](OCc4ccccc4)[C@H]3OCc3ccccc3)ccc2Cl)cc1]

wherein the process comprises deprotecting a compound of Formula IV

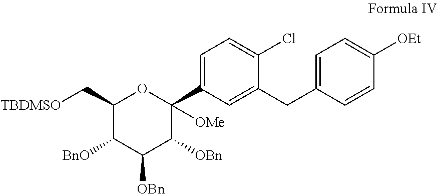 [SMILES: CCOc1ccc(Cc2cc([C@]3(OC)O[C@H](CO[Si](C)(C)C(C)(C)C)[C@@H](OCc4ccccc4)[C@H](OCc4ccccc4)[C@H]3OCc3ccccc3)ccc2Cl)cc1]

to obtain the compound of Formula V.

A ninth aspect of the present invention provides a process for the preparation of ertugliflozin of Formula I,

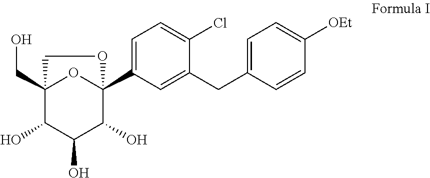 [SMILES: CCOc1ccc(Cc2cc([C@]34OC[C@](CO)(O3)[C@@H](O)[C@H](O)[C@H]4O)ccc2Cl)cc1]

wherein the process comprises:
a) deprotecting a compound of Formula IV

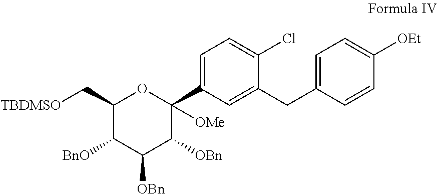 [SMILES: CCOc1ccc(Cc2cc([C@]3(OC)O[C@H](CO[Si](C)(C)C(C)(C)C)[C@@H](OCc4ccccc4)[C@H](OCc4ccccc4)[C@H]3OCc3ccccc3)ccc2Cl)cc1]

to obtain a compound of Formula V; and

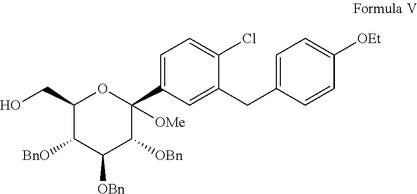 [SMILES: CCOc1ccc(Cc2cc([C@]3(OC)O[C@H](CO)[C@@H](OCc4ccccc4)[C@H](OCc4ccccc4)[C@H]3OCc3ccccc3)ccc2Cl)cc1]

b) converting the compound of Formula V to ertugliflozin of Formula I.

A tenth aspect of the present invention provides a process for the preparation of a compound Formula VII,

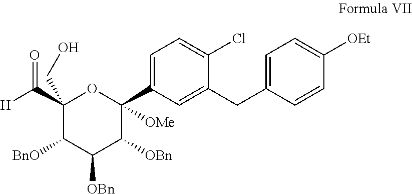 [SMILES: CCOc1ccc(Cc2cc([C@]3(OC)O[C@@](C=O)(CO)[C@@H](OCc4ccccc4)[C@H](OCc4ccccc4)[C@H]3OCc3ccccc3)ccc2Cl)cc1]

wherein the process comprises reacting a compound of Formula VI

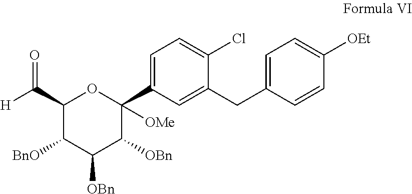 [SMILES: CCOc1ccc(Cc2cc([C@]3(OC)O[C@H](C=O)[C@@H](OCc4ccccc4)[C@H](OCc4ccccc4)[C@H]3OCc3ccccc3)ccc2Cl)cc1]

with formaldehyde in the presence of a base to obtain the compound of Formula VII.

An eleventh aspect of the present invention provides a process for the preparation of ertugliflozin of Formula I,

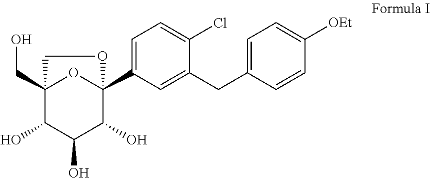 [SMILES: CCOc1ccc(Cc2cc([C@]34OC[C@](CO)(O3)[C@@H](O)[C@H](O)[C@H]4O)ccc2Cl)cc1]

wherein the process comprises:
a) reacting a compound of Formula VI

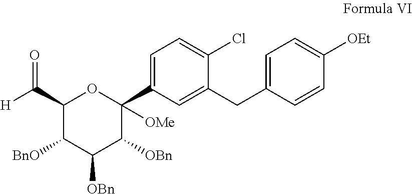 [SMILES: CCOc1ccc(Cc2cc([C@]3(OC)O[C@H](C=O)[C@@H](OCc4ccccc4)[C@H](OCc4ccccc4)[C@H]3OCc3ccccc3)ccc2Cl)cc1]

with formaldehyde in the presence of a base to obtain a compound of Formula VII; and

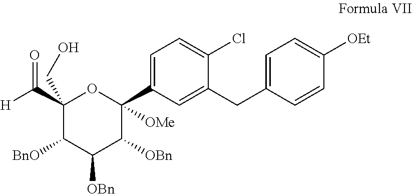 [SMILES: CCOc1ccc(Cc2cc([C@]3(OC)O[C@@](C=O)(CO)[C@@H](OCc4ccccc4)[C@H](OCc4ccccc4)[C@H]3OCc3ccccc3)ccc2Cl)cc1]

b) converting the compound of Formula VII to ertugliflozin of Formula I.

A twelfth aspect of the present invention provides a process for the preparation of a compound Formula VIII,

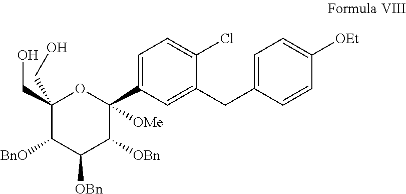

wherein the process comprises reducing a compound of Formula VII

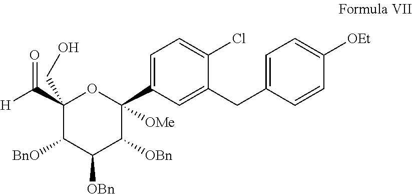 [SMILES: CCOc1ccc(Cc2cc([C@]3(OC)O[C@@](C=O)(CO)[C@@H](OCc4ccccc4)[C@H](OCc4ccccc4)[C@H]3OCc3ccccc3)ccc2Cl)cc1]

to obtain the compound of Formula VIII.

A thirteenth aspect of the present invention provides a process for the preparation of ertugliflozin of Formula I,

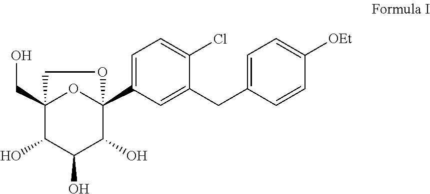 [SMILES: CCOc1ccc(Cc2cc([C@]34OC[C@](CO)(O3)[C@@H](O)[C@H](O)[C@H]4O)ccc2Cl)cc1]

wherein the process comprises:
a) reducing a compound of Formula VII

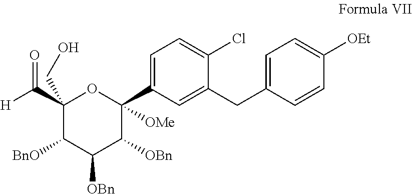 [SMILES: CCOc1ccc(Cc2cc([C@]3(OC)O[C@@](C=O)(CO)[C@@H](OCc4ccccc4)[C@H](OCc4ccccc4)[C@H]3OCc3ccccc3)ccc2Cl)cc1]

to obtain a compound of Formula VIII; and

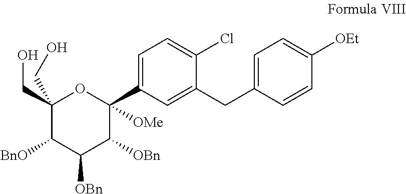

b) converting the compound of Formula VIII to ertugliflozin of Formula I.

A fourteenth aspect of the present invention provides a process for the preparation of a compound Formula VII,

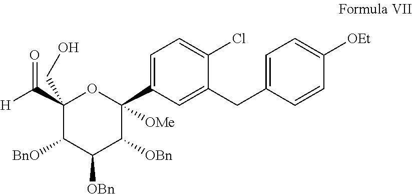 [SMILES: CCOc1ccc(Cc2cc([C@]3(OC)O[C@@](C=O)(CO)[C@@H](OCc4ccccc4)[C@H](OCc4ccccc4)[C@H]3OCc3ccccc3)ccc2Cl)cc1]

wherein the process comprises:
a) protecting a compound of Formula II

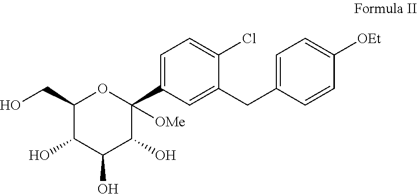 [SMILES: CCOc1ccc(Cc2cc([C@]3(OC)O[C@H](CO)[C@@H](O)[C@H](O)[C@H]3O)ccc2Cl)cc1]

with tert-butyldimethylsilyl chloride to obtain a compound of Formula III;

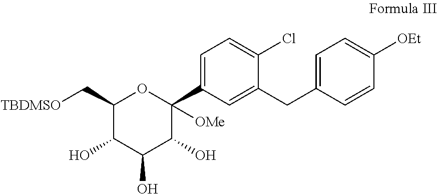 [SMILES: CCOc1ccc(Cc2cc([C@]3(OC)O[C@H](CO[Si](C)(C)C(C)(C)C)[C@@H](O)[C@H](O)[C@H]3O)ccc2Cl)cc1]

b) protecting the compound of Formula III with benzyl bromide to obtain a compound of Formula IV; and

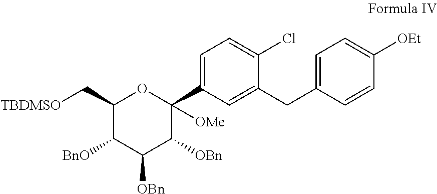 [SMILES: CCOc1ccc(Cc2cc([C@]3(OC)O[C@H](CO[Si](C)(C)C(C)(C)C)[C@@H](OCc4ccccc4)[C@H](OCc4ccccc4)[C@H]3OCc3ccccc3)ccc2Cl)cc1]

c) converting the compound of Formula IV to a compound of Formula VII.

A fifteenth aspect of the present invention provides a process for the preparation of ertugliflozin of Formula I,

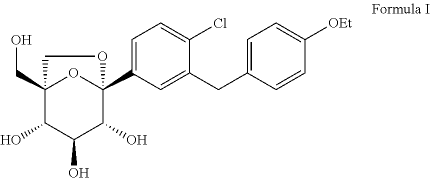 [SMILES: CCOc1ccc(Cc2cc([C@]34OC[C@](CO)(O3)[C@@H](O)[C@H](O)[C@H]4O)ccc2Cl)cc1]

wherein the process comprises:
a) protecting a compound of Formula II

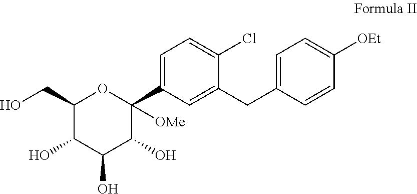 [SMILES: CCOc1ccc(Cc2cc([C@]3(OC)O[C@H](CO)[C@@H](O)[C@H](O)[C@H]3O)ccc2Cl)cc1]

with tert-butyldimethylsilyl chloride to obtain a compound of Formula III;

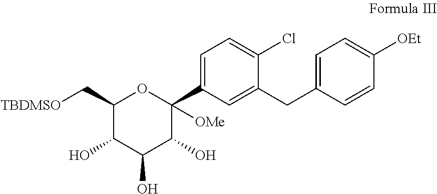 [SMILES: CCOc1ccc(Cc2cc([C@]3(OC)O[C@H](CO[Si](C)(C)C(C)(C)C)[C@@H](O)[C@H](O)[C@H]3O)ccc2Cl)cc1]

b) protecting the compound of Formula III with benzyl bromide to obtain a compound of Formula IV;

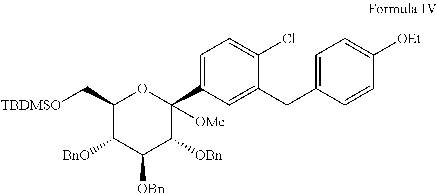 [SMILES: CCOc1ccc(Cc2cc([C@]3(OC)O[C@H](CO[Si](C)(C)C(C)(C)C)[C@@H](OCc4ccccc4)[C@H](OCc4ccccc4)[C@H]3OCc3ccccc3)ccc2Cl)cc1]

c) converting the compound of Formula IV to a compound of Formula VII; and

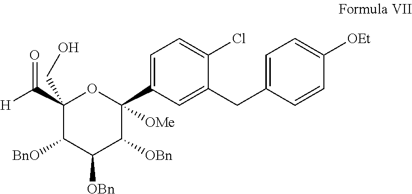 [SMILES: CCOc1ccc(Cc2cc([C@]3(OC)O[C@@](C=O)(CO)[C@@H](OCc4ccccc4)[C@H](OCc4ccccc4)[C@H]3OCc3ccccc3)ccc2Cl)cc1]

d) converting the compound of Formula VII to ertugliflozin of Formula I.

A sixteenth aspect of the present invention provides a compound of Formula III.

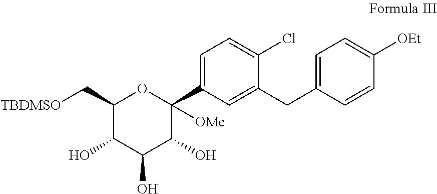 [SMILES: CCOc1ccc(Cc2cc([C@]3(OC)O[C@H](CO[Si](C)(C)C(C)(C)C)[C@@H](O)[C@H](O)[C@H]3O)ccc2Cl)cc1]

A seventeenth aspect of the present invention provides a compound of Formula IV.

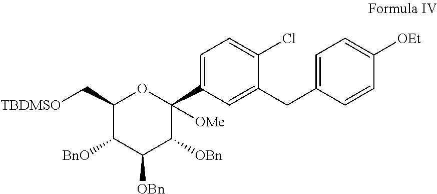 [SMILES: CCOc1ccc(Cc2cc([C@]3(OC)O[C@H](CO[Si](C)(C)C(C)(C)C)[C@@H](OCc4ccccc4)[C@H](OCc4ccccc4)[C@H]3OCc3ccccc3)ccc2Cl)cc1]

An eighteenth aspect of the present invention provides a compound of Formula VII.

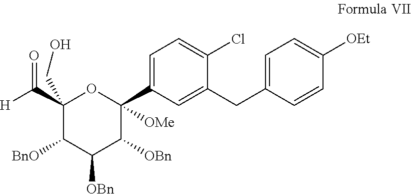 [SMILES: CCOc1ccc(Cc2cc([C@]3(OC)O[C@@](C=O)(CO)[C@@H](OCc4ccccc4)[C@H](OCc4ccccc4)[C@H]3OCc3ccccc3)ccc2Cl)cc1]

A nineteenth aspect of the present invention provides the use of compounds of Formula III, Formula IV, or Formula VII for the preparation of ertugliflozin of Formula I or ertugliflozin L-pyroglutamic acid of Formula Ia.

The present invention further provides the conversion of ertugliflozin of Formula I to ertugliflozin L-pyroglutamic acid of Formula Ia.

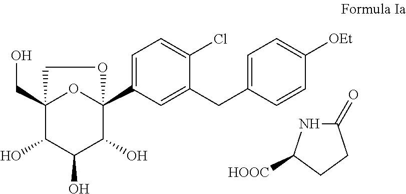

The compound of Formula II may be prepared by methods known in the art, for example, the methods described in U.S. Pat. No. 8,283,454 or by the method as described herein. The compound of Formula II may be isolated, or the reaction mixture containing the compound of Formula II may be used as such for the next step.

The protection of the compound of Formula II with tert-butyldimethylsilyl chloride to obtain the compound of Formula III is carried out in the presence of a base and a solvent. Preferably, the base is triethylamine. Preferably, the solvent is dichloromethane.

The protection of the compound of Formula II is carried out at a temperature of about 10° C. to about 40° C. Preferably, the protection is carried out at a temperature of about 25° C. to about 30° C.

The protection of the compound of Formula II is carried out for about 6 hours to about 20 hours. Preferably, the protection is carried out for about 10 hours to about 15 hours.

The compound of Formula III may be isolated by employing one or more techniques selected from the group consisting of filtration, decantation, extraction, distillation, evaporation, chromatography, precipitation, concentration, crystallization, centrifugation, and recrystallization. The compound of Formula III may further be dried using conventional techniques, for example, drying, drying under vacuum, spray drying, freeze drying, air drying, or agitated thin film drying, or the reaction mixture containing the compound of Formula III may be used as such for the next step.

The protection of the compound of Formula III with benzyl bromide to obtain the compound of Formula IV is carried out in the presence of a base and a solvent. Preferably, the base is sodium hydride. Preferably, the solvent is N,N-dimethylformamide.

The protection of a compound of Formula III with benzyl bromide is carried out at a temperature of about 10° C. to about 40° C. Preferably, the protection is carried out at a temperature of about 25° C. to about 30° C.

The protection of a compound of Formula III is carried out for about 1 hour to about 4 hours. Preferably, the protection is carried out for about 1 hour to about 2 hours.

The compound of Formula IV may be isolated by employing one or more techniques selected from the group consisting of filtration, decantation, extraction, distillation, evaporation, chromatography, precipitation, concentration, crystallization, centrifugation, and recrystallization. The compound of Formula IV may further be dried using conventional techniques, for example, drying, drying under vacuum, spray drying, freeze drying, air drying, or agitated thin film drying, or the reaction mixture containing the compound of Formula IV may be used as such for the next step.

The deprotection of the compound of Formula IV to obtain the compound of Formula V is carried out in the presence of an acid or an acid chloride, and a solvent.

The acid is selected from the group consisting of organic acids or inorganic acids.

Examples of organic acids include formic acid and acetic acid. Examples of inorganic acids include hydrochloric acid, sulphuric acid, nitric acid, and perchloric acid. The acid chloride is selected from the group consisting of acetyl chloride, oxalyl chloride, and thionyl chloride. Preferably, the acid chloride is acetyl chloride.

Preferably, the solvent is a mixture of methanol and dichloromethane.

The deprotection of the compound of Formula IV is carried out at a temperature of about 10° C. to about 40° C. Preferably, the deprotection is carried out at a temperature of about 25° C. to about 30° C.

The deprotection of the compound of Formula IV is carried out for about 30 minutes to about 2 hours. Preferably, the deprotection is carried out for about 1 hour.

The deprotection of the compound of Formula IV may also be carried out in the presence of a catalyst, for example, tetrabutylammonium fluoride.

The compound of Formula V may be isolated by employing one or more techniques selected from the group consisting of filtration, decantation, extraction, distillation, evaporation, chromatography, precipitation, concentration, crystallization, centrifugation, and recrystallization. The compound of Formula V may further be dried using conventional techniques, for example, drying, drying under vacuum, spray drying, freeze drying, air drying, or agitated thin film drying, or the reaction mixture containing the compound of Formula V may be used as such for the next step.

The oxidation of the compound of Formula V to obtain the compound of Formula VI is carried out with oxalyl chloride and dimethyl sulfoxide in the presence of a base and a solvent. Preferably, the base is triethylamine. Preferably, the solvent is dichloromethane.

The oxidation of the compound of Formula V is carried out at a temperature of about 10° C. to about 40° C. Preferably, the oxidation is carried out at a temperature of about 25° C. to about 30° C.

The oxidation of the compound of Formula V is carried out for about 1 hour to about 4 hours. Preferably, the oxidation is carried out for about 1 hour to about 2 hours.

The compound of Formula VI may be isolated by employing one or more techniques selected from the group consisting of filtration, decantation, extraction, distillation, evaporation, chromatography, precipitation, concentration, crystallization, centrifugation, and recrystallization. The compound of Formula VI may further be dried using conventional techniques, for example, drying, drying under vacuum, spray drying, freeze drying, air drying, or agitated thin film drying, or the reaction mixture containing the compound of Formula VI may be used as such for the next step.

The reaction of the compound of Formula VI with formaldehyde in the presence of a base to obtain the compound of Formula VII, the compound of Formula VIII, or the mixture thereof is carried out in a solvent at a temperature of about 10° C. to about 40° C. Preferably, the reaction is carried out at a temperature of about 25° C. to about 30° C. Preferably, the base is potassium hydroxide. Preferably, the solvent is N,N-dimethylformamide.

The amount of formaldehyde (37% aqueous solution) used for reacting the compound of Formula VI with formaldehyde is about 1 mole equivalent to about 5 mole equivalents with respect to the compound of Formula VI. Preferably, the amount of formaldehyde is about 2 mole equivalents to about 3 mole equivalents with respect to the compound of Formula VI.

The amount of the base used for reacting the compound of Formula VI with formaldehyde is about 1 mole equivalent to about 3 mole equivalents with respect to the compound of Formula VI. Preferably, the amount of the base is about 1 mole equivalent to about 1.5 mole equivalents with respect to the compound of Formula VI.

Preferably, the potassium hydroxide is dissolved in water and cooled to a temperature of about 15° C. to about 20° C. prior to the addition.

The reaction of the compound of Formula VI with formaldehyde is carried out for about 2 hours to about 10 hours. Preferably, the reaction is carried out for about 4 hours to about 8 hours.

The compound of Formula VII, the compound of Formula VIII, or the mixture thereof may be isolated by employing one or more techniques selected from the group consisting of filtration, decantation, extraction, distillation, evaporation, chromatography, precipitation, concentration, crystallization, centrifugation, and recrystallization. The compound of Formula VII, the compound of Formula VIII, or the mixture thereof may further be dried using conventional techniques, for example, drying, drying under vacuum, spray drying, freeze drying, air drying, or agitated thin film drying, or the reaction mixture containing the compound of Formula VII, the compound of Formula VIII, or the mixture thereof may be used as such for the next step.

The reduction of the compound of Formula VII to obtain the compound of Formula VIII is carried out in the presence of a reducing agent and a solvent. Preferably, the solvent is methanol.

The reducing agent is selected from the group consisting of sodium borohydride, lithium aluminum hydride, lithium borohydride, aluminum hydride, diisobutylaluminum hydride, Raney Nickel, and palladium/carbon. Preferably, the reducing agent is sodium borohydride.

The reduction of the compound of Formula VII is carried out at a temperature of about 10° C. to about 40° C. Preferably, the reduction is carried out at a temperature of about 20° C. to about 30° C.

The reduction of the compound of Formula VII is carried out for about 30 minutes to about 2 hours. Preferably, the reduction is carried out for about 1 hour.

The reaction of the compound of Formula VI with formaldehyde in the presence of a base to obtain the compound of Formula VIII is carried out in a solvent at a temperature of about 10° C. to about 40° C. Preferably, the reaction is carried out at a temperature of about 25° C. to about 30° C. Preferably, the base is potassium hydroxide. Preferably, the solvent is N,N-dimethylformamide.

The amount of formaldehyde (37% aqueous solution) used for reacting the compound of Formula VI with formaldehyde is about 10 mole equivalents to about 25 mole equivalents with respect to the compound of Formula VI. Preferably, the amount of formaldehyde is about 15 mole equivalents to about 20 mole equivalents with respect to the compound of Formula VI.

The amount of the base used for reacting the compound of Formula VI with formaldehyde is about 1 mole equivalent to about 5 mole equivalents with respect to the compound of Formula VI. Preferably, the amount of the base used is about 2 mole equivalents to about 4 mole equivalents with respect to the compound of Formula VI.

Preferably, the potassium hydroxide is dissolved in water and cooled to a temperature of about 15° C. to about 20° C. prior to the addition.

The reaction of the compound of Formula VI with formaldehyde is carried out for about 2 hours to about 10 hours. Preferably, the reaction is carried out for about 4 hours to about 8 hours.

The compound of Formula VIII may be isolated by employing one or more techniques selected from the group consisting of filtration, decantation, extraction, distillation, evaporation, chromatography, precipitation, concentration, crystallization, centrifugation, and recrystallization. The compound of Formula VIII may further be dried using conventional techniques, for example, drying, drying under vacuum, spray drying, freeze drying, air drying, or agitated thin film drying, or the reaction mixture containing the compound of Formula VIII may be used as such for the next step.

The cyclization of the compound of Formula VIII to obtain the compound of Formula IX is carried out in the presence of an acid and a solvent. Preferably, the solvent is dichloromethane.

The acid is selected from the group consisting of organic acids and inorganic acids. Examples of organic acids include formic acid, acetic acid, and trifluoroacetic acid. Examples of inorganic acids include hydrochloric acid, sulphuric acid, nitric acid, and perchloric acid. Preferably, the acid is trifluoroacetic acid.

The cyclization of the compound of Formula VIII is carried out at a temperature of about 10° C. to about 40° C. Preferably, the cyclization is carried out at a temperature of about 25° C. to about 30° C.

The cyclization of the compound of Formula VIII is carried out for about 2 hours to about 5 hours. Preferably, the cyclization is carried out for about 3 hours to about 4 hours.

The compound of Formula IX may optionally be isolated by employing one or more techniques selected from the group consisting of filtration, decantation, extraction, distillation, evaporation, chromatography, precipitation, concentration, crystallization, centrifugation, and recrystallization. The compound of Formula IX may further be dried using conventional techniques, for example, drying, drying under vacuum, spray drying, freeze drying, air drying, or agitated thin film drying.

The debenzylation of the compound of Formula IX to obtain ertugliflozin of Formula I is carried out in the presence of a hydrogenation agent, hydrogen gas, and a solvent. The hydrogenation agent is selected from the group consisting of palladium/carbon, platinum oxide, and Raney nickel. Preferably, the hydrogenation agent is palladium/carbon. Preferably, the solvent is a mixture of methanol and tetrahydrofuran.

The debenzylation of the compound of Formula IX is carried out in the presence of 1,2-dichlorobenzene.

The debenzylation of the compound of Formula IX is carried out at a hydrogen pressure, for example, of about 1.5 kg per cm² to about 4.0 kg per cm², preferably at a hydrogen pressure of about 3 kg per cm² to about 3.5 kg per cm².

The debenzylation of the compound of Formula IX is carried out at a temperature of about 10° C. to about 40° C. Preferably, the debenzylation is carried out at a temperature of about 25° C. to about 30° C.

The debenzylation of the compound of Formula IX is carried out for about 2 hours to about 6 hours. Preferably, the debenzylation is carried out for about 4 hours.

The ertugliflozin of Formula I may be isolated by employing one or more techniques selected from the group consisting of filtration, decantation, extraction, distillation, evaporation, chromatography, precipitation, concentration, crystallization, centrifugation, and recrystallization. The ertugliflozin of Formula I may further be dried using conventional techniques, for example, drying, drying under vacuum, spray drying, freeze drying, air drying, or agitated thin film drying.

The ertugliflozin of Formula I is optionally purified by dissolving ertugliflozin in methyl tert-butyl ether followed by the addition of n-hexane.

Further, ertugliflozin of Formula I may be converted into ertugliflozin L-pyroglutamic acid by following the process described in the art, for example, in U.S. Pat. No. 8,080,580.

While the present invention has been described in terms of its specific aspects, certain modifications and equivalents will be apparent to those skilled in the art and are intended to be included within the scope of the present invention.

The following examples are for illustrative purposes only and should not be construed as limiting the scope of the invention in any way.

### Methods:

NMR spectrum was recorded using a Bruker AVANCE III (400 MHz) NMR spectrometer.

The Mass spectrum was recorded using a MASS (API 2000) LC/MS/MS system, AB Sciex® QTRAP® LC/MS/MS system.

### Examples

### Example 1: Preparation of 3,4,5-tris[(trimethylsilyl)oxy]-6-{[(trimethylsilyl)oxy]methyl}-tetrahydro-2H-pyran-2-one

3,4,5-Trihydroxy-6-(hydroxymethyl)tetrahydro-2H-pyran-2-one (100 g) was dissolved in tetrahydrofuran (1 L) to obtain a solution. N-Methylmorpholine (455.9 g) was added to the mixture, and the reaction mixture was cooled to 0° C. to 5° C. Trimethylsilyl chloride (366 g) was added to the reaction mixture, and then the mixture was stirred for 15 hours at 35° C. to 40° C. The reaction mixture was cooled to 0° C. to 5° C. Ethyl acetate (1 L) and deionized water (1 L) were added, and then the layers were separated. The organic layer was washed with deionized water (1 L) and an aqueous sodium chloride solution (5%, 1 L), and then concentrated under reduced pressure. The residue obtained was dissolved in toluene (200 mL), then filtered through a Hyflo®, and then concentrated under reduced pressure. The residue was again dissolved in toluene (200 mL), and then concentrated under reduced pressure to afford the title compound.

### Yield: 295 g

### Example 2: Preparation of (5-bromo-2-chlorophenyl)(4-ethoxyphenyl)methanone

5-Bromo-2-chloro benzoic acid (100 g) was dissolved in dichloromethane (200 mL) to obtain a solution. N,N-Dimethylformamide (1 mL) and oxalyl chloride (91.6 g) were added to the reaction mixture, and then the mixture was stirred for 15 hours at 25° C. to 30° C. The reaction mixture was concentrated under reduced pressure at 40° C. to 45° C. Dichloromethane (80 mL) was added to the mixture, and then the mixture was cooled to 0° C. to 5° C. Ethyl phenyl ether (51.9 g) and aluminum chloride (64 g) were added to the mixture, and then the mixture was stirred for 2 hours at 0° C. to 10° C. The reaction mixture was poured into chilled water (600 mL) maintained at 0° C. to 5° C., and then stirred for 60 minutes. Deionized water (200 mL) and dichloromethane (350 mL) were added to the mixture, and then the layers were separated. The aqueous layer was extracted with dichloromethane (350 mL). The combined organic layers were washed with aqueous hydrochloric acid (1 L, 100 mL hydrochloric acid in 900 mL deionized water), an aqueous sodium hydroxide solution (4%, 1 L), and an aqueous sodium chloride solution (20%, 1 L), successively. The organic layer was concentrated under reduced pressure to obtain an oily residue (115 g). Ethanol (250 mL) was added to the oily residue, then the mixture was stirred for 5 minutes, then deionized water (100 mL) was added to the mixture, and then the mixture was stirred for 60 minutes at 20° C. to 25° C. The mixture was filtered, and the wet solid obtained was washed with a mixture of deionized water (120 mL) and ethanol (60 mL). Ethanol (250 mL) was added to the wet solid and the slurry obtained was stirred for 5 minutes. Deionized water (100 mL) was added to the mixture over 20 minutes, and then the mixture was stirred for 60 minutes at 20° C. to 25° C. The solid was filtered, then washed with a mixture of water (120 mL) and ethanol (60 mL) and then dried under reduced pressure at 40° C. to 45° C. for 12 hours to 15 hours to afford the title compound.

### Yield: 96 g

### Example 3: Preparation of 4-bromo-1-chloro-2-(4-ethoxybenzyl)benzene

Aluminum chloride (176.6 g) was added to dimethyl ether (900 mL) at 0° C. to 5° C. to obtain a reaction mixture. Sodium borohydride (50 g) was added to the mixture in lots over 30 minutes at 0° C. to 5° C., and then the mixture was stirred for 30 minutes at 0° C. to 5° C. A solution of (5-bromo-2-chlorophenyl)(4-ethoxyphenyl)methanone (225 g, Example 2) in dimethyl ether (450 mL) was added slowly at 0° C. to 25° C. The temperature of the reaction mixture was raised to 60° C. to 65° C., and then the mixture was stirred for 30 hours. The reaction mixture was concentrated under reduced pressure at 40° C. to 45° C., and the obtained residue was cooled to 0° C. to 5° C. The obtained residue was slowly added into precooled deionized water (2250 mL) at 0° C. to 10° C. Ethyl acetate (2250 mL) was added to the mixture, and then the mixture was stirred for 10 minutes. The layers were separated, and the aqueous layer was extracted with ethyl acetate (450 mL). The combined organic layers were washed with an aqueous sodium bicarbonate solution (8%, 1125 mL) and deionized water (450 mL), and then concentrated under reduced pressure at 40° C. to 45° C. Methanol (675 mL) was added to the mixture, then the mixture was cooled to 0° C. to 5° C., then stirred for 1 hour at 0° C. to 5° C., and then the solid was filtered. The wet solid obtained was washed with precooled methanol (75 mL), and then dried under vacuum at 40° C. to 45° C. for 12 hours to 15 hours to afford the title compound.

### Yield: 178 g

### Example 4: Preparation of methyl-1-C-[4-chloro-3-(4-ethoxybenzyl)phenyl]-α-D-glucopyranoside (Formula II)

A mixture of tetrahydrofuran (400 mL) and toluene (600 mL) was cooled to −75° C. to −70° C. n-Butyllithium (160 mL, 2.3 M) was slowly added at −75° to −60° C. over 30 minutes. A solution of 4-bromo-1-chloro-2-(4-ethoxybenzyl)benzene (100 g dissolved in 300 mL toluene, Example 3) was added at −75° C. to −60° C. over 30 minutes, and then the mixture was stirred for 30 minutes at −75° C. to −70° C. A solution of 3,4,5-tris[(trimethylsilyl)oxy]-6-{[(trimethylsilyl)oxy]methyl}tetrahydro-2H-pyran-2-one (65.6 g, Example 1) in toluene (700 mL) was added at −75° C. to −60° C. over 60 minutes, and then the mixture was stirred for 2 hours at −75° C. to −70° C. A solution of methane sulfonic acid (30 mL) in methanol (670 mL) was added at −75° C. to −70° C. over 60 minutes. The reaction mixture was warmed to 20° C. to 25° C., and was then stirred for 18 hours. An aqueous sodium bicarbonate solution (8%, 500 mL) and ethyl acetate (500 mL) were added to the reaction mixture. The layers were separated, and the aqueous layer was extracted with ethyl acetate (2×500 mL). The combined organic layers were washed with an aqueous sodium chloride solution (5%, 500 mL), and then concentrated under reduced pressure at 40° C. to 45° C. The residue was dissolved in toluene (200 mL), and then hexane (700 mL) was added under nitrogen. The mixture was stirred for 30 minutes, then filtered under nitrogen, and then washed with hexane (100 mL) to obtain a wet solid. The wet solid was dried under reduced pressure at 35° C. to 40° C. for 12 hours to 15 hours to afford the title compound.

### Yield: 82 g

### Example 5: Preparation of methyl 6-O-[tert-butyl(dimethyl)silyl]-1-C-[4-chloro-3-(4-ethoxybenzyl)phenyl]-α-D-glucopyranoside (Formula III)

Methyl-1-C-[4-chloro-3-(4-ethoxybenzyl)phenyl]-α-D-glucopyrano side (75 g, Formula II, Example 4) was dissolved in dichloromethane (750 mL) to obtain a solution. Triethylamine (86.43 g) was added to the solution, and then the mixture was cooled to 10° C. to 15° C. tert-Butyldimethylsilyl chloride (30.9 g) was added to the mixture at 10° C. to 15° C., and then the mixture was stirred for 12 hours to 15 hours at 25° C. to 30° C. An aqueous ammonium chloride solution (20%, 750 mL) was added to the mixture, the mixture was stirred for 10 minutes to 15 minutes, and then the layers were separated. The organic layer was washed with deionized water (375 mL) and an aqueous sodium chloride solution (20%, 375 mL), successively. The organic layer was concentrated under reduced pressure at 40° C. to 45° C. and the residue was as such used for the next step.

¹H NMR (400 MHz, CDCl₃): δ ppm 0.09 (s, 3H), 0.11 (s, 3H), 0.91 (s, 12H), 1.39 (t, J=8 Hz, 3H), 3.06 (s, 3H), 3.20-3.22 (m, 1H), 3.63-3.66 (m, 2H), 3.87-3.96 (m, 7H), 6.80 (d, J=11.6 Hz, 2H), 7.07 (d, J=8.64, 2H), 7.30 (dd, J₁=8.32 Hz, J₂=2.1 Hz, 1H), 7.34 (s, 1H), 7.37 (t, J=2.04 Hz, 1H)

Mass (m/z): 570.4 (M+NH₄)⁺

### Example 6: Preparation of methyl 2,3,4-tri-O-benzyl-6-O-[tert-butyl(dimethyl)silyl]-1-C-[4-chloro-3-(4-ethoxybenzyl)phenyl]-α-D-glucopyranoside (Formula IV)

Methyl 6-O-[tert-butyl(dimethyl)silyl]-1-C-[4-chloro-3-(4-ethoxybenzyl)phenyl]-α-D-glucopyranoside (Formula III, Example 5) was dissolved in N,N-dimethylformamide (300 mL), and then the mixture was cooled to 0° C. to 5° C. Sodium hydride (33.9 g) was added to the mixture, and then the mixture was stirred for 30 minutes. Benzyl bromide (118.6 g) was added to the mixture at 0° C. to 25° C., and then the mixture was stirred for 2 hours at 25° C. to 30° C. The mixture was cooled to 0° C. to 5° C., and then an aqueous ammonium chloride solution (5%, 1500 mL) and ethyl acetate (750 mL) were added. The layers were separated, and the aqueous layer was extracted with ethyl acetate (750 mL). The combined organic layers were washed with deionized water (750 mL), and then concentrated under reduced pressure at 40° C. to 45° C. The obtained residue was used as such for the next step.

¹H NMR (400 MHz, CDCl₃): δ ppm 0.01 (s, 3H), 0.03 (s, 3H), 0.85 (s, 12H), 1.3 (t, J=8 Hz, 3H), 2.98 (s, 3H), 3.23 (d, J=9.5 Hz, 1H), 3.60 (m, 1H), 3.70-3.91 (m, 7H), 3.97-4.10 (m, 2H), 4.43 (d, J=10.6 Hz, 1H), 4.65 (d, J=10.8 Hz, 1H), 4.81-4.84 (m, 3H), 6.66 (d, J=8.7 Hz, 2H), 6.92-6.98 (m, 4H), 7.08-7.13 (m, 3H), 7.15-7.28 (m, 12H), 7.38-7.40 (m, 1H)

Mass (m/z): 840.6 (M+NH₄)⁺

### Example 7: Preparation of methyl 2,3,4-tri-O-benzyl-1-C-[4-chloro-3-(4-ethoxybenzyl)phenyl]-α-D-glucopyranoside (Formula V)

Methyl 2,3,4-tri-O-benzyl-6-O-[tert-butyl(dimethyl)silyl]-1-C-[4-chloro-3-(4-ethoxybenzyl)phenyl]-α-D-glucopyranoside (Formula IV, Example 6) was dissolved in a mixture of methanol (750 mL) and dichloromethane (75 mL). Acetyl chloride (13.6 g) was added to the mixture at 25° C. to 30° C., and then the mixture was stirred for 30 minutes. Deionized water (750 mL) was added to the mixture, and then the layers were separated. The organic layer was concentrated under reduced pressure at 40° C. to 45° C., and the residue was used as such for the next step.

### Example 8: Preparation of methyl 2,3,4-tri-O-benzyl-1-C-[4-chloro-3-(4-ethoxybenzyl)phenyl]-α-D-gluco-hexodialdo-1,5-pyranoside (Formula VI)

Oxalyl chloride (43.3 g) was added to dichloromethane (750 mL), and then the mixture was cooled to −80° C. to −75° C. A solution of dimethyl sulfoxide (40 g) in dichloromethane (375 mL) was slowly added to the mixture, and then the mixture was stirred at −80° C. to −75° C. for 30 minutes. A solution of methyl 2,3,4-tri-O-benzyl-1-C-[4-chloro-3-(4-ethoxybenzyl)phenyl]-α-D-glucopyranoside (Formula V, Example 7) in dichloromethane (375 mL) was slowly added at −80° C. to −65° C. over 30 minutes, and then the mixture was stirred for 2 hours. Triethylamine (86.4 g) was added at −80° C. to −65° C., then the temperature was raised to 25° C. to 30° C., and then the mixture was stirred for 2 hours. Aqueous hydrochloric acid (10%, 750 mL) was added, and then the layers were separated. The organic layer was washed with deionized water (750 mL), and concentrated under reduced pressure at 40° C. to 45° C. The obtained residue was used as such for the next step.

### Example 9: Preparation of methyl 2,3,4-tri-O-benzyl-1-C-[4-chloro-3-(4-ethoxybenzyl)phenyl]-5-(hydroxymethyl)-α-D-gluco-hexodialdo-1,5-pyranoside (Formula VII)

Methyl 2,3,4-tri-O-benzyl-1-C-[4-chloro-3-(4-ethoxybenzyl)phenyl]-α-D-gluco-hexodialdo-1,5-pyranoside (Formula VI, Example 8) was dissolved in N,N-dimethylformamide (750 mL), and then the mixture was cooled to 10° C. to 15° C. An aqueous formaldehyde solution (37%, 150 mL) was added to the mixture at 10° C. to 15° C., followed by the addition of an aqueous potassium hydroxide solution (4.8 g in 75 mL water) at 15° C. to 20° C. The temperature of the reaction mixture was raised to 25° C. to 30° C., and then the mixture was stirred for 8 hours. An aqueous sodium chloride solution (10%, 3750 mL) and ethyl acetate (750 mL) were added to the mixture, and then the layers were separated. The aqueous layer was extracted with ethyl acetate (750 mL). The combined organic layers were washed with deionized water (2×750 mL), and then the organic layer was concentrated under reduced pressure at 40° C. to 45° C. The obtained residue was used as such for the next step.

### Example 10: Preparation of methyl 2,3,4-tri-O-benzyl-1-C-[4-chloro-3-(4-ethoxybenzyl)phenyl]-5-(hydroxymethyl)-α-D-glucopyranoside (Formula VIII)

Methyl 2,3,4-tri-O-benzyl-1-C-[4-chloro-3-(4-ethoxybenzyl)phenyl]-5-(hydroxymethyl)-α-D-gluco-hexodialdo-1,5-pyranoside (Formula VII, Example 9) was dissolved in methanol (750 mL). Sodium borohydride (9.7 g) was slowly added to the mixture at 20° C. to 25° C., and then the mixture was stirred for 1 hour. The mixture was concentrated under reduced pressure at 40° C. to 45° C. Ethyl acetate (750 mL) and deionized water (750 mL) were added to the obtained residue, then the layers were separated, and then the aqueous layer was extracted with ethyl acetate (375 mL). The combined organic layers were washed with deionized water (750 mL), and then concentrated under reduced pressure at 40° C. to 45° C. The obtained residue was used as such for the next step.

### Example 11: Preparation of methyl 2,3,4-tri-O-benzyl-1-C-[4-chloro-3-(4-ethoxybenzyl)phenyl]-5-(hydroxymethyl)-α-D-glucopyranoside (Formula VIII)

Methyl 2,3,4-tri-O-benzyl-1-C-[4-chloro-3-(4-ethoxybenzyl)phenyl]-α-D-gluco-hexodialdo-1,5-pyranoside (Formula VI, Example 8, 5 g equivalent of Formula II) was dissolved in N,N-dimethylformamide (50 mL), and then the mixture was cooled to 10° C. to 15° C. An aqueous formaldehyde solution (37%, 18.5 mL) was added to the mixture at 10° C. to 15° C., followed by the addition of an aqueous potassium hydroxide solution (1.3 g in 6.5 mL water) at 15° C. to 20° C. The temperature of the reaction mixture was raised to 25° C. to 30° C., and then the mixture was stirred for 5 hours. An aqueous sodium chloride solution (10%, 250 mL) and ethyl acetate (250 mL) were added to the mixture, and then the layers were separated. The aqueous layer was extracted with ethyl acetate (50 mL). The combined organic layers were washed with deionized water (2×125 mL), and then the organic layer was concentrated under reduced pressure at 40° C. to 45° C. to afford the title compound.

### Example 12: Preparation of {(1S,2S,3S,4R,5S)-2,3,4-tris(benzyloxy)-5-[4-chloro-3-(4-ethoxybenzyl)phenyl]-6,8-dioxabicyclo[3.2.1]oct-1-yl}methanol (Formula IX)

Methyl 2,3,4-tri-O-benzyl-1-C-[4-chloro-3-(4-ethoxybenzyl)phenyl]-5-(hydroxymethyl)-α-D-glucopyranoside (Formula VIII, Example 10) was dissolved in dichloromethane (750 mL), and then the mixture was cooled to −10° C. to −5° C. to obtain a reaction mixture. Trifluoroacetic acid (38.9 g) was slowly added to the mixture, and then the mixture was stirred at −10° C. to −5° C. for 1 hour. The temperature was raised to 25° C. to 30° C., and then the mixture was stirred for 4 hours. An aqueous sodium bicarbonate solution (8%, 750 mL) was added to the mixture, and then the mixture was stirred for 10 minutes to 15 minutes. The layers were separated, and the aqueous layer was extracted with dichloromethane (375 mL). The combined organic layers were concentrated under reduced pressure at 40° C. to 45° C. The residue thus obtained was purified by column chromatography using ethyl acetate-hexane. The oily mass obtained was further purified by crystallization in ethyl acetate (25 mL) and hexane (400 mL) to afford the title compound.

### Yield: 18.5 g

### Example 13: Preparation of (1S,2S,3S,4R,5S)-5-{4-chloro-3-[(4-ethoxyphenyl)methyl]phenyl}-1-(hydroxymethyl)-6,8-dioxabicyclo[3.2.1]octane-2,3,4-triol (Formula I)

{(1S,2S,3S,4R,5S)-2,3,4-Tris(benzyloxy)-5-[4-chloro-3-(4-ethoxybenzyl)phenyl]-6,8-dioxabicyclo[3.2.1]oct-1-yl}methanol (18 g, Formula IX, Example 12) was dissolved in a mixture of methanol (270 mL) and tetrahydrofuran (270 mL) to obtain a reaction mixture. 1,2-Dichlorobenzene (9 mL) and palladium/carbon (10%, 5.44 g) were added to the reaction mixture, and then the mixture was twice flushed with 2 kg per cm² hydrogen gas pressure. The reaction mixture was agitated under hydrogen gas pressure (3 kg per cm² to 3.5 kg per cm²) at 25° C. to 30° C. for 6 hours. The mixture was filtered through a Hyflo®, and then the filtrate was concentrated under reduced pressure at 40° C. to 45° C. to obtain a residue. The residue was dissolved in ethyl acetate (180 mL), and then washed with an aqueous sodium chloride solution (10%, 2×180 mL). The organic layer was concentrated under reduced pressure at 40° C. to 45° C. to afford the title compound.

### Yield: 13.5 g

### Example 14: Purification of (1S,2S,3S,4R,5S)-5-{4-chloro-3-[(4-ethoxyphenyl)methyl]phenyl}-1-(hydroxymethyl)-6,8-dioxabicyclo[3.2.1]octane-2,3,4-triol (Formula I)

(1S,2S,3S,4R,5S)-5-{4-Chloro-3-[(4-ethoxyphenyl)methyl]phenyl}-1-(hydroxymethyl)-6,8-dioxabicyclo[3.2.1]octane-2,3,4-triol (11 g, Formula I, Example 13) was dissolved in methyl tert-butyl ether (11 mL), and then n-hexane (88 mL) was added slowly to the mixture at 25° C. to 30° C. The reaction mixture was stirred for 3 hours, then filtered, and then washed with n-hexane (44 mL) to obtain a wet solid. The wet solid was dried under reduced pressure at 25° C. to 30° C. for 12 hours to afford the title compound.

### Yield: 9.0 g

1-53. (canceled)

54. A compound selected from a compound of Formula III, a compound of Formula IV, or a compound of Formula VII.

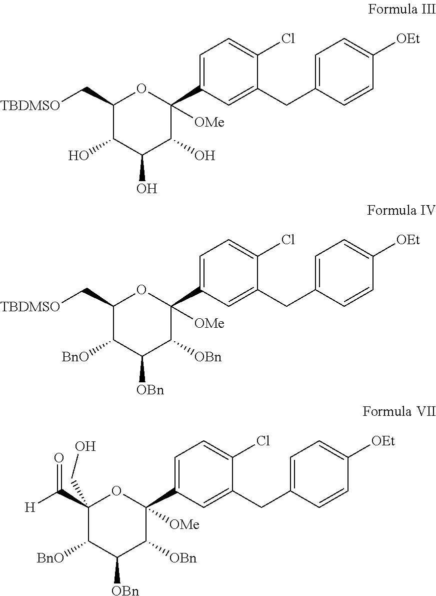 [SMILES: CCOc1ccc(Cc2cc([C@]3(OC)O[C@H](CO[Si](C)(C)C(C)(C)C)[C@@H](O)[C@H](O)[C@H]3O)ccc2Cl)cc1 ; CCOc1ccc(Cc2cc([C@]3(OC)O[C@H](CO[Si](C)(C)C(C)(C)C)[C@@H](OCc4ccccc4)[C@H](OCc4ccccc4)[C@H]3OCc3ccccc3)ccc2Cl)cc1 ; CCOc1ccc(Cc2cc([C@]3(OC)O[C@@](C=O)(CO)[C@@H](OCc4ccccc4)[C@H](OCc4ccccc4)[C@H]3OCc3ccccc3)ccc2Cl)cc1]

55. The compound of Formula III according to claim 54, which is prepared by a process that comprises protecting a compound of Formula II 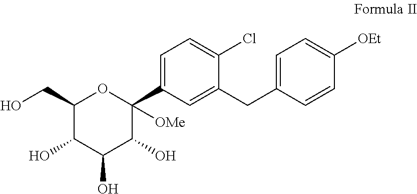 [SMILES: CCOc1ccc(Cc2cc([C@]3(OC)O[C@H](CO)[C@@H](O)[C@H](O)[C@H]3O)ccc2Cl)cc1]
with tert-butyldimethylsilyl chloride.

56. The compound of Formula III according to claim 55, which is further converted to ertugliflozin.

57. The compound of Formula IV according to claim 54, which is prepared by a process that comprises protecting a compound of Formula III with benzyl bromide.

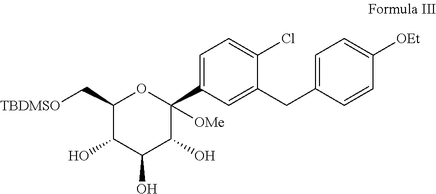 [SMILES: CCOc1ccc(Cc2cc([C@]3(OC)O[C@H](CO[Si](C)(C)C(C)(C)C)[C@@H](O)[C@H](O)[C@H]3O)ccc2Cl)cc1]

58. The compound of Formula IV according to claim 57, which is further converted to ertugliflozin.

59. The compound of Formula IV according to claim 54, which is prepared by a process that comprises:
a) protecting a compound of Formula II 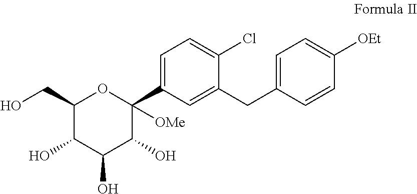 [SMILES: CCOc1ccc(Cc2cc([C@]3(OC)O[C@H](CO)[C@@H](O)[C@H](O)[C@H]3O)ccc2Cl)cc1]
with tert-butyldimethylsilyl chloride to obtain a compound of Formula III; and 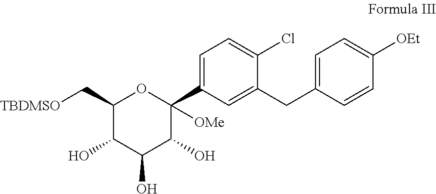 [SMILES: CCOc1ccc(Cc2cc([C@]3(OC)O[C@H](CO[Si](C)(C)C(C)(C)C)[C@@H](O)[C@H](O)[C@H]3O)ccc2Cl)cc1]
b) protecting the compound of Formula III with benzyl bromide to obtain the compound of Formula IV.

60. The compound of Formula IV according to claim 59, which is further converted to ertugliflozin.

61. The compound of Formula VII according to claim 54, which is prepared by a process that comprises reacting a compound of Formula VI with formaldehyde in the presence of a base.

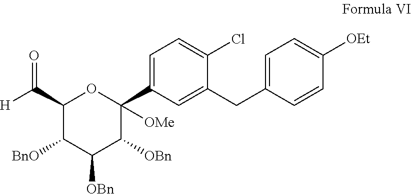 [SMILES: CCOc1ccc(Cc2cc([C@]3(OC)O[C@H](C=O)[C@@H](OCc4ccccc4)[C@H](OCc4ccccc4)[C@H]3OCc3ccccc3)ccc2Cl)cc1]

62. The compound of Formula VII according to claim 61, which is further converted to ertugliflozin.

63. The compound of Formula IV according to claim 54, which is further converted to a compound of Formula V 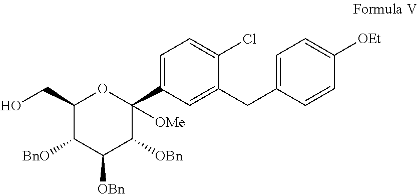 [SMILES: CCOc1ccc(Cc2cc([C@]3(OC)O[C@H](CO)[C@@H](OCc4ccccc4)[C@H](OCc4ccccc4)[C@H]3OCc3ccccc3)ccc2Cl)cc1]
by deprotecting the compound of Formula IV.

64. The compound of Formula IV according to claim 54, which is converted to a compound of Formula V by deprotecting the compound of Formula IV, 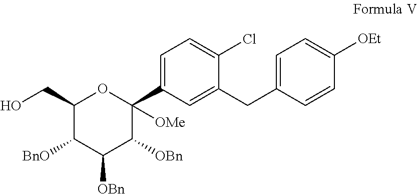 [SMILES: CCOc1ccc(Cc2cc([C@]3(OC)O[C@H](CO)[C@@H](OCc4ccccc4)[C@H](OCc4ccccc4)[C@H]3OCc3ccccc3)ccc2Cl)cc1]
and further converting the compound of Formula V into ertugliflozin.

65. The compound of Formula VII according to claim 54, which is further converted to a compound of Formula VIII by reducing the compound of Formula VII.

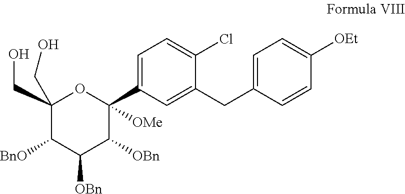

66. The compound of Formula VII according to claim 54, which is converted to a compound of Formula VIII by reducing the compound of Formula VII, 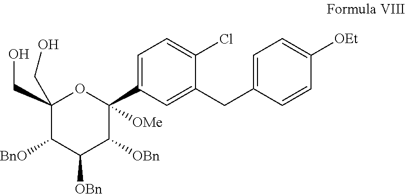
and further converting the compound of Formula VIII into ertugliflozin.

67. The compound of Formula VII according to claim 54, which is prepared by a process that comprises:
a) protecting a compound of Formula II 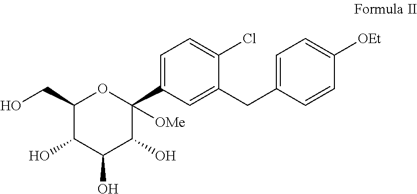 [SMILES: CCOc1ccc(Cc2cc([C@]3(OC)O[C@H](CO)[C@@H](O)[C@H](O)[C@H]3O)ccc2Cl)cc1]
with tert-butyldimethylsilyl chloride to obtain a compound of Formula III; 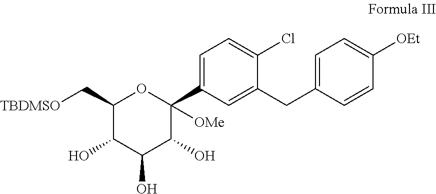 [SMILES: CCOc1ccc(Cc2cc([C@]3(OC)O[C@H](CO[Si](C)(C)C(C)(C)C)[C@@H](O)[C@H](O)[C@H]3O)ccc2Cl)cc1]
b) protecting the compound of Formula III with benzyl bromide to obtain a compound of Formula IV; and 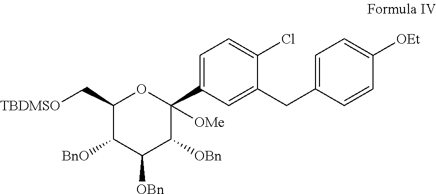 [SMILES: CCOc1ccc(Cc2cc([C@]3(OC)O[C@H](CO[Si](C)(C)C(C)(C)C)[C@@H](OCc4ccccc4)[C@H](OCc4ccccc4)[C@H]3OCc3ccccc3)ccc2Cl)cc1]
c) converting the compound of Formula IV to a compound of Formula VII.

68. The compound of Formula VII according to claim 67, which is further converted to ertugliflozin.
